(19) United States
(12) Patent Application Publication
(10) Pub. No.: US 2017/0188896 A1
(43) Pub. Date: Jul. 6, 2017

(54) SYSTEM FOR CONTROLLING THE ACCOMMODATION DEMAND DETECTION IN AN ARTIFICIAL ACCOMMODATION SYSTEM

(71) Applicant: Karlsruher Institut fuer Technologie (KIT), Karlsruhe (DE)

(72) Inventors: Helmut GUTH, Eggenstein-Leopoldshafen (DE); Ulrich GENGENBACH, Remchingen (DE); Georg BRETTHAUER, Karlsruhe (DE); Jan ROTHMUND, Schwaebisch Hall (DE); Liane KOKER, Stutensee (DE); Joerg NAGEL, Eggenstein-Leopoldshafen (DE); Thomas MARTIN, Karlsruhe (DE); Christoph BECK, Esslingen-Liebersbronn (DE)

(21) Appl. No.: 15/307,005

(22) PCT Filed: Apr. 28, 2015
(86) PCT No.: PCT/EP2015/000868

§ 371 Date: Jan. 16, 2017

(30) Foreign Application Priority Data
Apr. 30, 2014 (DE) 10 2014 106 036.9

(51) Int. Cl. A61B 5/11; G06K 9/00; G06F 3/01; A61B 5/16; A61B 3/18; A61B 3/10; A61B 3/113; A61B 8/10; A61B 3/14

(52) U.S. Cl. CPC A61B 5/1123; A61B 8/10; G06K 9/0061; G06F 3/013; A61B 3/14; A61B 3/18; A61B 3/1015; A61B 3/113; A61B 5/1103; A61B 5/1101; A61B 5/161; A61F 2009/00846

(57) ABSTRACT

A control system for controlling an accommodation demand detection in an ophthalmic technical system includes at least one sensor configured to detect a signal sequence from a body itself or an environmental signal sequence and to convert the detected signal sequence into measurement signals, and at least one detector configured to convert the measurement signals into a control signal that influences the accommodation demand detection. The detector includes a signal processor configured to compare the measurement signals with a reference signal bandwidth and to generate the control signal When the measurement signals are covered by a predetermined reference signal bandwidth.

### CROSS REFERENCE TO RELATED APPLICATIONS

This application is a U.S. National Stage Application under 35 U.S.C. §371 of International Application No. PCT/EP2015/000868 filed on Apr. 28, 2015, and claims benefit to German Patent Application No. DE 10 2014 106 036.9 filed on Apr. 30, 2014. The International Application was published in German on Nov. 5, 2015 as WO 2015/165584 A1 under PCT Article 21(2).

### FIELD

The invention relates to a control system for controlling, preferably triggering, detection of the accommodation demand in an ophthalmic technical system, in particular an artificial accommodation system. The control system is used for influencing the ophthalmic technical system according to demand using the body's own conscious or unconscious signals, or environmental effects that do not have to be directly useful for detecting an accommodation demand. Furthermore, the invention relates to a contact lens including a control system for controlling detection of the accommodation demand in an ophthalmic technical system.

### BACKGROUND

The human eye is a natural optical system that forms sharp images of objects on the retina using a number of refractive boundaries. If the distance from the object being viewed changes, the imaging behavior of the optical system also has to change in order to form images with the same sharpness on the retina. In the human eye, this takes place by deforming the lens using the ciliary muscle (musculus ciliaris), the shape and position of the front and rear of the lens substantially changing as a result (accommodation).

An artificial accommodation system is an ophthalmic technical system (OTS), i.e. an artificial optical system that comprises a lens system having at least one optical lens of adjustable focal length. This system stands out because it is either placed in the eye (e.g. as an implant) or is in close contact with ocular tissue or ocular fluids (placed on the eye or between the eyes and other body tissues). As described for example in DE 10 2005 038 542 A1, said system comprises, in addition to an adjustable lens system, an information acquisition system, an information processing system, an energy supply system and an attachment system. The information acquisition system is used to detect measurement signals, from which the information processing system determines an accommodation demand, and these signals are relayed to actuators in the lens system as actuation signals. The accommodation demand is a variable that is necessary in a natural control loop as a control variable for the lens system in order to adjust a particular focal length for an eye to focus on a sighted object. In the natural eye, determining the accommodation demand is an integral part of the ocular motor function, with the ciliary muscle acting as an actuator for adjusting the refractive power of the lens, but can also be used as an information source for said measurement signals.

As described for example in DE 10 2005 038 542 A1, by implanting an artificial accommodation system as an autonomous implant in the human eye, it is possible to restore the accommodation capacity loss as a result of age (presbyopia) or a cataract (grey star) operation. The system is intended to be placed completely in the capsular sac of the human eye (instead of the natural lens).

In addition, U.S. Pat. No. 6,851,805 B2 and US 2009/0015785 A1, for example, disclose smart contact lenses in which the accommodation system is integrated in a supplementary lens for the eye, such as spectacles or a contact lens directly on the eyeball.

U.S. Pat. No. 6,851,805 B2 describes an accommodating contact lens having integrated sensors and an integrated power supply. The detection of the accommodation demand is not triggered, but instead influences and eye positions are directly associated with the specific accommodation demand.

To detect the viewing direction and the activity of the user, a tilt sensor (tilt switch) and a gyroscope are provided, for example. The sensor signals are relayed to actuators to adjust the focal length. The concept does not allow for communication between two contact lenses, or for the detection of complex movement patterns of the user. The accommodation demand is determined solely and directly on the basis of the measured values from the sensors of the relevant lens.

By way of example, US 2009/0015785 A1 discloses a lens system (intraocular lens, contact lens, corneal inlay or glasses) comprising concentric annular lenses, the refractive power of which can be adjusted and which can be controlled and adjusted according to the ambient light or pupil width. To detect the ambient light, photodetectors integrated in the lens are proposed in particular. In this system too, detection of the accommodation demand is only determined by the sensors of the relevant lens system.

Said systems use sensors that directly or indirectly generate control signals for an adjustable lens system. The signals thus represent the accommodation demand, i.e. the individual signal values are each linked by mathematical relationships to a focal length to be set and/or to another optical setting in the lens system.

In addition, US 2012/0140167 A1 describes a contact lens and an intraocular lens system having two focal lengths that can be adjusted by means of various alternative actuator concepts. The switching preferably takes place on the basis of detected eyelid movements, preferably using photosensors integrated in the system that detect shadows from the lids. Alternatively, by means of acceleration sensors or distance meters, eye movements are also intended for causing switching. To determine the accommodation demand, the optional use of a distance meter is also proposed.

Accommodation demand detection should be distinguished from accommodation demand. Accommodation demand detection includes activating generation of measurement data and evaluating the signals with a view to deducing an accommodation demand therefrom. Said detection controls the detection or generation of the signals, preferably measurement signals, that are required for the necessary accommodation demand in order to adjust the lens system.

### SUMMARY

In an embodiment, the present invention provides a control system for controlling an accommodation demand detection in an ophthalmic technical system. The control system includes at least one sensor configured to detect a signal sequence from a body or an environmental signal sequence and to convert the detected signal sequence into measurement signals, and at least one detector configured to convert the measurement signals into a control signal that influences the accommodation demand detection. The at least one detector comprises a signal processor configured to compare the measurement signals with a reference signal bandwidth and to generate the control signal when the measurement signals are covered by a predetermined reference signal bandwidth.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIGS. 1a and 1b are block diagrams of components of a controller for controlling the accommodation demand detection for one eye according to an embodiment of the invention;

FIG. 2 shows a circuit for detecting a signal sequence from the body itself according to an embodiment of the invention;

FIG. 3 shows a first binocular design for both eyes having two detection systems and bidirectional data exchange according to an embodiment of the invention; and

FIGS. 4a and b show further binocular designs for both eyes and a common detection system according to an embodiment of the invention.

### DETAILED DESCRIPTION

It is not known to influence the accommodation demand detection in an artificial accommodation system by means of external influences or the user's behavior. For example, the systems do not distinguish whether detection of said accommodation demand per se actually appears necessary. Typically, the detection is carried out continuously at predetermined intervals, for example. However, this not only entails higher energy consumption, but also means that the user may feel some discomfort with the system constantly readjusting.

In an embodiment, the invention provides a control system, e.g. a controller, for controlling the detection of the accommodation demand in an artificial accommodation system or other ophthalmic technical system (OTS), which allows significant amounts of energy to be saved in the accommodation system (or other OTS) without noticeably restricting the user, thereby allowing user comfort to be increased.

In an embodiment, a controller for controlling the detection of the accommodation demand in an artificial accommodation system is provided that intervenes in the control loop of an accommodation system by detecting body signals and/or environmental signals and using the measurement signals produced thereby to trigger, stop or otherwise influence detection of the accommodation demand. The measurement signals are converted to control signals, preferably to trigger signals, for detecting the accommodation demand for the accommodation system or OTS, i.e. are not converted to control signals for adjusting an accommodation demand of a lens system. During this conversion, the measurement signals are detected and associated with predetermined behavior patterns of the wearer of the accommodation system or OTS. According to this association, a likewise predetermined control signal is generated for detecting the accommodation demand.

The control signals are preferably trigger signals for activating or deactivating an accommodation demand detection or accommodation demand detection sequence in said control loop of the accommodation system (or OTS), for activating or deactivating an accelerated or decelerated clock sequence for the accommodation demand detection, or for stopping the accommodation demand detection. This controls the accommodation system (or OTS) according to the demand with a view to saving energy, increasing the service life or improving user comfort.

Therefore, the control signals are used to adjust or initiate operating states, in particular:
a. triggering an accommodation demand detection,
b. properties of the accommodation demand detection (e.g. frequency or frequency change of the demand measurement),
c. any control of an accommodation demand detection (e.g. switching on, off or over).

Unlike in known systems, the control signals do not include any signals for directly adjusting a particular focal length in the accommodation system. They are used to adjust and switch between operating states, and to change accommodation system operating parameters relevant for the process. For example, an operating state involves fixing or initiating an adjusted or predetermined focal length until the controller triggers cancellation of this operating state or triggers another operating state. Control is required, for example, when a system is to be switched on or off or when switching between different operating modes is to take place, e.g. the change initiated by the user from distance vision to close vision (comparable to tilting the head when wearing varifocal lenses) and vice versa.

A controller according to an embodiment of the invention comprises at least one sensor for detecting a signal sequence from the body itself or an environment signal sequence and for converting said sequence into measurement signals, and at least one detector for converting the measurement signals into a control signal that influences the accommodation demand detection.

The sensor is used to pick up signals, i.e. a system that allows for the physical detection of eye or blinking movements or environmental signals. Eye movements can be characterized using the variables position, speed and acceleration of the eyes in relation to a fixed reference system based on the head. Suitable sensors for detecting one or more of these variables are, for example, acceleration sensors, angular rate sensors, magnetic field sensors or optical sensors. Blinking movements can be quantitatively characterized inter alia by the degree of opening of the eyes, for which preferably capacitive, tactile or optical sensor concepts are used.

A blink is preferably detected by a switching process that only differentiates between an open and a closed lid and, for example, disregards the tiredness of the lens system wearer or ambient brightness. In this respect, just one sensor is often sufficient for the detection.

The detection of the ambient brightness is required, for example, for weighting a depth of field range and thus indirectly also an accommodation demand detection, which is not possible by detecting blinking alone.

By contrast, the degree of opening of the blink is dependent on said disregarded influences and in principle is also detected by the aforementioned sensor concepts. However, it is necessary to detect the different lid positions with greater differentiation, which, as the detection accuracy increases, also requires a greater number of individual sensors per lid, arranged as an array, a row and/or at various positions on the lid.

The detector comprises a signal processor that compares the measurement signals with reference signal bandwidths representative of said behavior patterns of the wearer of the accommodation system or OTS, recognizes this behavior pattern when the measurement signals are covered by a predetermined reference signal bandwidth, and generates a control signal.

Unlike a simple threshold, a reference signal bandwidth does not just have one value, but instead indicates a bandwidth in the sense of a tolerance range around a value for the body's own signals and/or environmental signals that is to be expected in an ideal situation, i.e. around a reference signal. For each possible control signal, an associated reference signal bandwidth that includes the idealized signal (reference signal) and the appropriate related tolerance range is defined. The received measurement signals are compared with the values included in the reference signal bandwidth. If a measurement signal is completely within the value range spanned by the reference signal bandwidth, the corresponding reference signal is recognized and the control signal belonging to the reference signal is generated by the detector. In the process, the body's own signals and/or environmental signals expected are preferably also signal sequences that develop over time (reference signal sequence), preferably in the sense of signal patterns. The reference signal bandwidth should not be confused with a bandwidth in the frequency range, but rather it corresponds to a preferably exact reference signal sequence that is expanded by a tolerance range around this reference signal sequence that preferably also develops over time. Together with the tolerance range that changes over time, the reference signal sequence forms a chronologically developing reference signal bandwidth. The tolerance range allows valid measurement signals and measurement signal sequences to be detected when there are disturbances, noise and variability caused by non-ideal environmental conditions or non-ideal execution of patterns. Therefore, the reference signal or reference signal sequence and the measurement signal or measurement signal sequence do not have to be completely congruent, but in fact only have to be congruent to a minimum level to be set. The reference signal bandwidths thus represent the set of all the reference signals and reference signal sequences expanded by their respective tolerance ranges.

A preferred signal detection embodiment involves pattern detection having a trigger for triggering a control signal, with the measurement signals optionally segmented in advance.

The segmentation is an optional first signal processing step that can be used to divide a measurement signal, e.g. an acceleration signal caused by an eye movement, into individual characteristic segments, e.g. an acceleration segment and a deceleration segment. The segmentation can be replaced, for example, when using decision networks based on training data (e.g. artificial neural networks).

By detecting the pattern, it is possible to interpret the measurement signals and/or, where applicable, the segments in relation to predefined movement patterns. In the process, the measurement signals are compared with a preferably chronologically developing reference signal bandwidth, and the control signal is generated if the measurement signals are covered by a predetermined reference signal bandwidth. Therefore, in this signal processing step, a distinction is drawn between natural movements and movements that the user consciously makes for the purpose of control. The trigger is used to assign a movement pattern to a defined control action, and thus performs the task of event triggering. The control action implements the control command. The result of the control action is the control signal, e.g. the adjustment of an operating state or change in operating parameters.

The invention is based on the field of man-machine interaction. As a system for interaction between man and machine, the invention makes use of the body's own signals and/or environmental signals detected in the eye or directly adjacent to the eye.

The system for interaction between man and machine preferably detect conscious or unconscious eye movements or everyday behavior patterns of the wearer of the accommodation system or OTS that are interpreted and classified in the signal processor of the detector, and the control signal is generated on the basis of this classification. A control signal is either a one-off signal or a signal sequence.

Unconscious eye movements are preferably saccade-like and reflex blinking movements. Conscious eye movements are preferably conscious movements and movement sequences of the eyes and/or lids. They also in particular include complete closure of the eyes or unnatural eye movements, e.g. blinking in time codes (e.g. Morse codes). They preferably stimulate trigger signals for detecting the accommodation demand, the term “trigger signal” including both single trigger signals and trigger signal sequences within the scope of the application.

Everyday behavior patterns represent typical activities or actions of the wearer of the accommodation system or OTS that occur in recurring patterns and can also be recognized on this basis. Typical behavior patterns are, for example, reading, computer work, watching television, physical sports such as running, driving, housework, waiting, observing, and other activities when the desired focal length of the eye does not change or only changes to a small extent unifocally, when it is switched back and forth bifocally between two focal lengths, or when no particular focus adjustment is required (e.g. during sleep). Any necessary minor changes to the desired focal length are preferably within the depth of field range and covered thereby. Therefore, these focal lengths are preferably implemented in the accommodation system as fixed settings, i.e. the accommodation demand does not have to be re-determined each time one of these settings is repeated. As a result, the control signal includes an actuation signal (trigger signal) for either a predetermined focal length range or a predetermined, preferably reduced frequency in the accommodation demand detection in the accommodation system. Switching back and forth between two focal lengths can be triggered, for example, by an additional control signal that by a blink or characteristic eye movement, even without accommodation demand detection.

Environmental influences are external influences, for example darkness, that are represented by an environmental signal sequence. The control signal preferably includes an actuation signal (trigger signal) for either a predetermined focal length range or a predetermined, preferably reduced frequency in the accommodation demand detection in the accommodation system.

Specifically, the following behavior patterns and systems are advantageous for detecting said patterns and for controlling the accommodation demand detection in an artificial accommodation system (or OTS):

1. Unconscious Eye Movements:

Unconscious eye movements include unconscious saccades such as eyeball rotations or eyelid closure movements. Saccades are eye movements in which the eyeball preferably rotates. Changes to the accommodation demand are generally accompanied by saccade movements of the eye. Saccades occur at an average frequency of less than 10 Hz, preferably of from 0.2 to 4 Hz (saccade length typically approximately 50-250 ms). Using the example of an eye rotation, saccades have an initial rotational acceleration of approximately 20000 deg/s², regardless of the amplitude. On a circular path about the eye center using an assumed radius of approximately 10 mm (distance of the implant from the center), this produces a tangential acceleration of approximately 1 g (simple gravity acceleration).

The eyeball movements can be detected as the body's own signal sequences, preferably by acceleration sensors that are preferably on the surface of the eyeball. These sensors either receive the acceleration movement directly, or alternatively indirectly via gyroscopes or magnetic field sensors relative to a stationary system, e.g. magnetic field system.

Acceleration sensors are preferably conventional inertial sensors and, for use in the invention, are preferably piezoelectric, capacitive or inductive sensors, and more preferably sensors that can be miniaturized photolithographically or produced using silicon technology.

However, accelerations that are too slow, such as slow sequential movements, can lead to incorrect measurements and, for example, do not stimulate any saccade-based triggering either. In this case it is proposed that, in the absence of suitable acceleration signals or signal sequences caused by saccades or control signals such as trigger signals, a predetermined low frequency of from 0.25 to 2.0 Hz, preferably between 0.5 and 1.5 Hz, in the accommodation detection is automatically set in the accommodation system since only slight differences in the accommodation demand can be assumed between two saccades.

In addition, unconscious eye movements also include spontaneous lid closure movements (e.g. blinking). Unconscious blinking movements are generally accompanied by saccades (movements for changing the field of view) since visual perception is suppressed anyway during saccades. Lid closure movements can preferably be detected by light-sensitive sensors that are preferably arranged on the eye, on a contact lens for example, and are covered by the moving lid depending on the position thereof. One simple way of detecting lid closures requires just one sensor that only detects whether the lid is closed, but not the exact lid position. If the exact lid position or the lid closure speed is desired, at least two sensors that are covered in succession when the lid closes are required. The sensitivity is within the preferred visible wavelength range of the eye, preferably 400-500 nm. In light-sensitive sensors (photosensors), however, environmental influences (e.g. flickering, flashes, etc.) can be misinterpreted as blinking. To counter this, a maximum sensor scanning frequency limit of e.g. approximately 10 Hz (preferably between 5 and 100 Hz) is proposed.

2. Everyday Behavior Patterns

Everyday behavior patterns include cases in which the eye and/or the lid undergo either a characteristic load sequence or movement sequence. In all of them, the accommodation demand only varies to an insignificant extent over a particular time period, i.e. there is no need to detect the accommodation demand. In the process, preselected focal length ranges are preferably repeated, fixed for a longer period of time, or switched back and forth between two or more predetermined focal lengths. These behavior patterns in particular include reading, working in front of a screen, watching television, driving, rest periods, or sport such as jogging.

During reading, characteristic sudden movement patterns having a great number of saccade movements occur, e.g. when reading a line towards the right followed by a large saccade to the left at the end of each line, without there being any need for a large change in the refractive power of the accommodation system. As long as this saccade pattern is detected, the accommodation demand does not have to be adjusted. The control signal is preferably used as an adjustment signal for a predetermined focal length range (e.g. depth of field range around a predetermined focal length), or alternatively as a trigger for a one-time or repeated detection of the accommodation demand for adjusting this focal length range. As an alternative, the control signal is used as a start signal for setting a reduced frequency to approximately 0.5 Hz for the accommodation demand detection in the accommodation system (or OTS). Acceleration sensors on the eyeball for detecting eyeball rotations when the viewing direction changes are preferably used as the sensor.

As when reading, very characteristic eye movements having lots of saccades also occur when searching, and specifically normally in very quick succession but without a defined direction, unlike reading. Saccades only briefly stop occurring when a point is being observed in the space and is blurry. In this respect, searching exercises are a separate behavior pattern that can preferably be detected by an acceleration sensor on the eyeball, and the control signal preferably stimulates detection of the accommodation demand for adjusting a focal length range to be maintained afterwards. In an alternative, sensors are provided, and the evaluation and reaction to searching movements are carried out according to a design proposed above for saccades.

As when reading, just one adjustment of the focal length in the accommodation system (or OTS) is required over a relatively long period of time e.g. when working in front of a screen, watching television, driving, when in the theater or cinema, or when wandering around or observing. A largely unchanged viewing direction, in particular relative to the vertical, is also characteristic of these behavior patterns. Preferably, acceleration sensors alone or supplemented by other sensors are suitable for detecting this behavior pattern. If the position and viewing direction of the accommodation system wearer relative to the surroundings do not change or only change insignificantly (e.g. when working in front of a screen, watching television, when in the theater or cinema, or when observing), these other sensors include magnetic field sensors or gyroscopes, but also light and color sensors for the images or objects being observed in each case. If the position and/or the viewing direction changes constantly or significantly, e.g. when driving, these other sensors are preferably limited to light and color sensors that in particular detect greater light and/or color changes. These changes occur in some situations that often require particular attention, e.g. when driving into a tunnel or in the event of glare, when switching lights on and off, the end of a film, or other external influences. These particular situations can be taught to or stored in the system or taken into account in the permissible reference signal bandwidth (learning system). If these other sensors operate independently of the acceleration sensor, they generate an additional measurement signal and preferably also a control signal that triggers one instance of one or more accommodation demand detections and, where applicable, checks the predetermined adjusted focal length range. Furthermore, light and color sensors are suitable for detecting certain wavelength spectra or optical pulse sequences that are characteristic, for example, of certain computer or television screens when in operation. Otherwise, the focal length setting generally only has to be changed when leaving or stopping this behavior pattern, for example when looking at the clock or a speedometer.

Another group of behavior patterns includes activities in which the eye as a whole follows an acceleration pattern within a reference signal bandwidth, even without rotation in the eye socket. This group includes, for example, a number of individual and team sports, e.g. jogging, swimming, cycling, ball games, or basically any other activity (e.g. hiking, operating any kind of motor vehicle or vibrating tools or equipment) in which the head undergoes a cyclic or otherwise characteristic acceleration sequence. In one cycle for example, this acceleration pattern is defined by both the step frequency (preferably between 1 and 3 Hz) and the acceleration towards the gravitational field at an increased amplitude of >1 g. The aforementioned acceleration sensors, of which the measurement signals are compared with the characteristic reference signal bandwidths in the signal processor of the detector, are suitable sensors for detecting these acceleration patterns, and the control signal is stimulated when the measurement signals are covered by one of these reference signal bandwidths. Preferably, the control signal in the form of a trigger signal stimulates activation or deactivation of an accommodation demand detection. If deactivated, the adjusted focal length and the focal length range around said length (depth of field range around the focal length) are preferably fixed in the accommodation system, or alternatively said range is preferably adjusted in the distant range.

3. Conscious Eye and Blinking Movements

Conscious eye movements include the cases in which the eye and/or the lid perform a characteristic movement or movement sequence that is consciously brought about at a freely selectable time, i.e. not as a reflex to a state brought about consciously or unconsciously. This includes in particular conscious blinks or sequences of blinks and/or conscious rotation of the eyeballs.

Preferably, these movements or movement sequences can be sufficiently distinguished from the aforementioned unconscious eye movements or everyday behavior patterns or even from environmental signals. They are preferably distinguished by an unnatural movement sequence of the eyeball and/or the lids, e.g. by a specific blinking time sequence, similar to a simple Morse code, in one eye or both, or by initiating certain eyeball orientations.

Blinks or blinking sequences can be used to activate or deactivate detection of the accommodation demand and to indirectly fix, adjust or change the refractive power of the accommodation system, for example also for indirectly switching between near vision and distance vision. In one embodiment, it is also possible to use the control signal to activate switching states that allow focal length adjustments, e.g. a focal length change in predetermined stages, with the accommodation demand detection deactivated, each stage being triggered separately by a control signal. Using blinks or blinking sequences, it is also possible to control certain operating states such as automatic accommodation measurement, a standby mode, an increase and/or reduction of either the measurement frequency or the accommodation demand detection, as well as simply switching the accommodation system on and off.

Conscious blinks and blinking sequences are detected by the light-sensitive sensors or switches, as described above in the context of unconscious blinks. Within the meaning of the invention, blinks and blinking sequences are both monocular and binocular. In one possible embodiment, Morse codes or another sequence of conscious blinking movements are divided in any manner between both eyes. Evaluating binocular signals requires the use of a communication path between the systems in both eyes. In the process, the communication path is preferably activated in a pulsed manner immediately after a blink or blinking sequence occurs.

Conscious eye closure, which, as described above, can be detected by light-sensitive sensors or switches preferably located on the eyeball (or inside the eye), is preferably suitable for deactivating the accommodation demand detection or the entire accommodation system when the eyes are closed. The deactivation is preferably carried out following an adjustable time period once a closed lid has been detected, preferably usually after 2 to 5 seconds.

The deactivation of the accommodation demand detection ends with reactivation, preferably immediately when light is detected again, e.g. when the eyelids are opened or in the dark when light can first be detected. Closed eyelids are preferably detected by said light-sensitive sensors or switches, it being particularly advantageous to analyze the blue light spectrum (approximately 420-480 mm wavelength, corresponding to approximately 624-714 THz frequency). In the blue light spectrum, the transmission of the eyelid is at its lowest, while red and infrared light can pass through a lid particularly effectively. In this respect, detection of blue light components is preferably suitable for detecting a closed eye, for detecting said eye movements and thus for activating and deactivating the accommodation demand detection as described above.

Conscious eye movements further include unnatural eye movements caused consciously, e.g. squinting or eyeball rotations (eye path movements), in particular when the lids are closed. As described above, these can preferably be detected using acceleration sensors or magnetic field sensors attached to the eyeball. If squinting (corresponding to a calculated accommodation demand of over 3 dpt, preferably over 10 dpt) or extreme turns of the eyes preferably relative to one another (vergence angle measurement) or relative to the head, they stimulate a trigger signal for a function in the accommodation system, e.g. a reduction in the frequency for detecting the accommodation demand.

During eye path movements when the lids are open or closed, or even in combination with a blinking time sequence, the system wearer performs an eye movement along a predetermined path or according to a predetermined pattern that can be detected by the sensors and recognized by the signal processor by being compared with a reference signal bandwidth. The control signal generated therefrom is used as a trigger for a function in the accommodation system. An example predetermined eye path could start with a look above to the left (a.l.). Next, the view wanders down to the right (d.r.), and then ends down to the left (d.l.). In principle, the number of positions in the path can be variably set and can be so high as to prevent any risk of confusion with natural eye movements.

4. ENVIRONMENTAL SIGNAL SEQUENCE

The group of environmental signal sequences includes the cases in which the detectable signals are not generated by movements of the eyes and/or lids, or not only by movements thereof, but rather are produced or influenced by external signals. These in particular include certain characteristic wavelengths (e.g. certain colors of light, e.g. of a television or a computer screen) or light intensities from the surroundings down to darkness (e.g. at night). The light intensities are either detected as a whole, or individual wavelength ranges are detected selectively, preferably by light-sensitive sensors or switches (e.g. photosensors, photodiodes, CCD chips, etc.) that may or may not have a filter and are preferably arranged directly on the surface of the eyeball. In one embodiment, the sensors or switches are integrated in a contact lens, the detected light shining through the contact lens material, which thus acts as an optical filter.

If, when the eyelid is open or closed, the detected light intensity drops below a predeterminable limit intensity as night approaches or the environment is darkening for another reason, this is recognized by the signal processor of the detector and a control signal or a control signal sequence is generated. This control signal preferably sets a rest state, i.e. the accommodation demand detection is stopped or set to a lower repetition frequency. In another embodiment, in addition to the detection being stopped, a fixed focal length is set. If the detected light intensity then exceeds the limit intensity again, the rest state is terminated by another control signal being generated.

Alternatively, when characteristic light colors are detected, e.g. like when watching television, when in the cinema, working on a computer or driving, control signals for preferably one preset focal length can be generated in the accommodation system. When watching television, for example, the viewer is exposed to a very characteristic light progression owing to the cuts in the film. Generally, the cuts are accompanied by a jump in brightness. In films, a cut occurs on average every 1 to 10 seconds. Within one sequence, between two steps, a continuous brightness progression is produced by the changing image. The color temperature of a television image is mostly blue. This results from selecting the color space sRGB that has a white point at a color temperature of 6500 K (difference and distinguishing criterion from daylight—approximately 5000-5800 K) for the image display. In general, the color temperature can be used for recognizing an environmental signal sequence.

The invention preferably also covers a complete system, i.e. an accommodation system having at least one of said controller for controlling the accommodation demand detection. For this purpose, the detector is preferably connected to the information processing system of the artificial accommodation system via the signal processor, and transmits the control signal via this connection in order to influence the accommodation demand detection and thus the control loop for controlling accommodation.

The accommodation system is preferably an accommodating intraocular lens (IOL), i.e. an implant for substituting or complementing the artificial lens or a contact lens system that is placed on an eye as an accommodation system. In particular when the accommodation system is an implant and the controller for controlling the accommodation demand detection is for example placed on the eye, e.g. via a contact element or contact lens outside the eyeball, said connection for transmitting the control signal is preferably produced on the basis of optical or electromagnetic transmission paths or a wireless connection.

The invention further covers a use of said controller for controlling the accommodation demand detection for an accommodation system, and a method for controlling the accommodation demand detection. In the method, a signal sequence from the body itself or an environmental signal sequence are in particular detected by at least one sensor and converted into measurement signals thereby. The measurement signals are then preferably compared with a reference signal bandwidth in a signal processor in a detector, and the control signal is generated when the measurement signals are covered by a predetermined reference signal bandwidth.

The system can be monocular or binocular. In a binocular system, one system is positioned on each eye, the systems preferably communicating with one another wirelessly and in particular exchanging or combining measured values and signals for controlling the accommodation demand detection, as well as dividing up tasks.

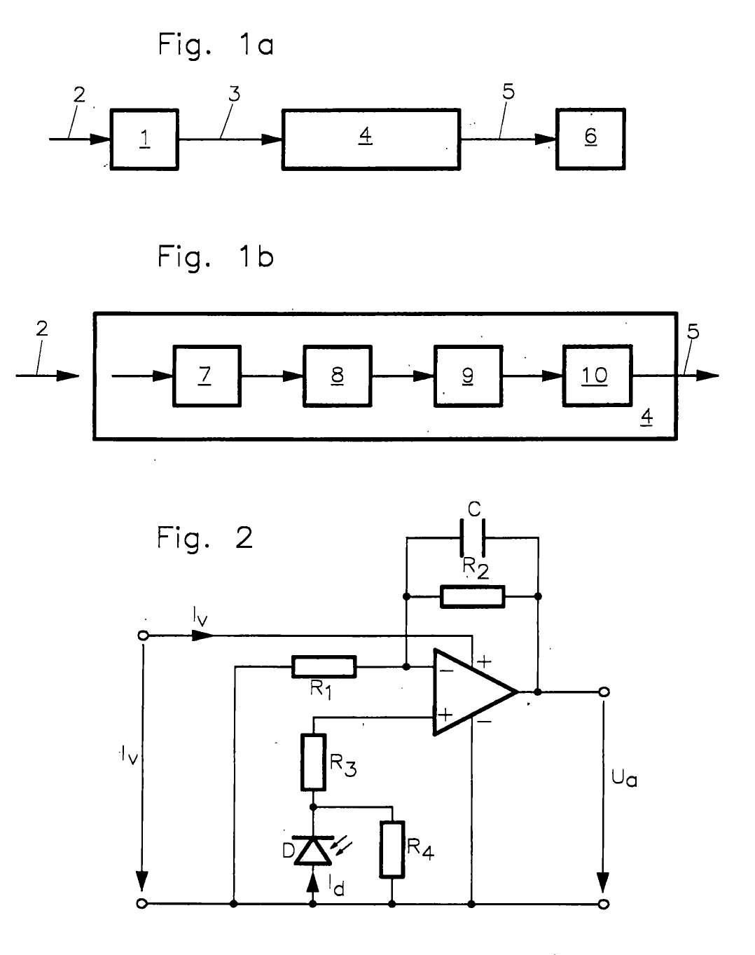

In an embodiment shown in FIG. 1a, the controller for controlling the accommodation demand detection include a sensor 1 (signal receiver) for detecting a signal sequence 2 and converting it into measurement signals 3. These are supplied to a detector 4 and processed therein to form a control signal 5 for influencing an accommodation system 6 or an OTS. Preferably, all of said components are integrated in the accommodation system or OTS, either as an implant or integrated in a smart contact lens and preferably without any other external components.

FIG. 1b shows the circuit diagram of the detector 4 in detail. The measurement signals 3 are first preferably supplied to segmentation 7. The segmentation is an optional first signal processing step, by which the eye and blinking movements are broken down into individual characteristic segments, preferably time segments. The segmentation makes it simpler for the subsequent signal processing since the segmentation can optionally limit signal recognition to particular segments. The effort and thus energy requirements for the signal processing are thus reduced advantageously. Alternatively, training data-based decision networks (e.g. artificial neural networks) enable integral signal processing, without the need for a segmentation step as a separate prior step.

The measurement signals are sent (directly) to a signal processor 8 in segmented or unsegmented form. The signal processor makes it possible to interpret the segments in relation to predefined movement patterns by the systems comparing the measurement signals with a reference signal bandwidth and detecting when the measurement signals are covered by a predetermined reference signal bandwidth. In particular, in this signal processing step, a distinction is drawn between natural movements and movements that the user performs consciously for control purposes. If a movement pattern is recognized, it is relayed to a trigger 9 as a signal. The trigger is used to assign a movement pattern to a defined control action, and thus performs the task of event triggering for a control action 10. The control action implements the control command, i.e. the generation of the control signal 5, e.g. for controlling the detection of the accommodation demand.

In terms of the signal receiver, FIG. 2 shows an amplifier circuit, by way of example, for detecting a signal sequence from the body itself, e.g. lid closure. The circuit is distinguished by the use of an ultra-low-power operation amplifier as both a low-pass filter and a pre-amplifier for the light sensor used (photodiode), and by way of these features the circuit allows not only for a low overall size, but also low energy requirements.

Furthermore, in addition to monocular usage (detection of signals in one eye), the systems and a method for controlling the accommodation demand detection can also be used in a binocular manner (detection of signals in both eyes) in an artificial accommodation system or OTS. For example, blinking patterns that include a combined blink and/or rotational movements of both eyes can thus be detected.

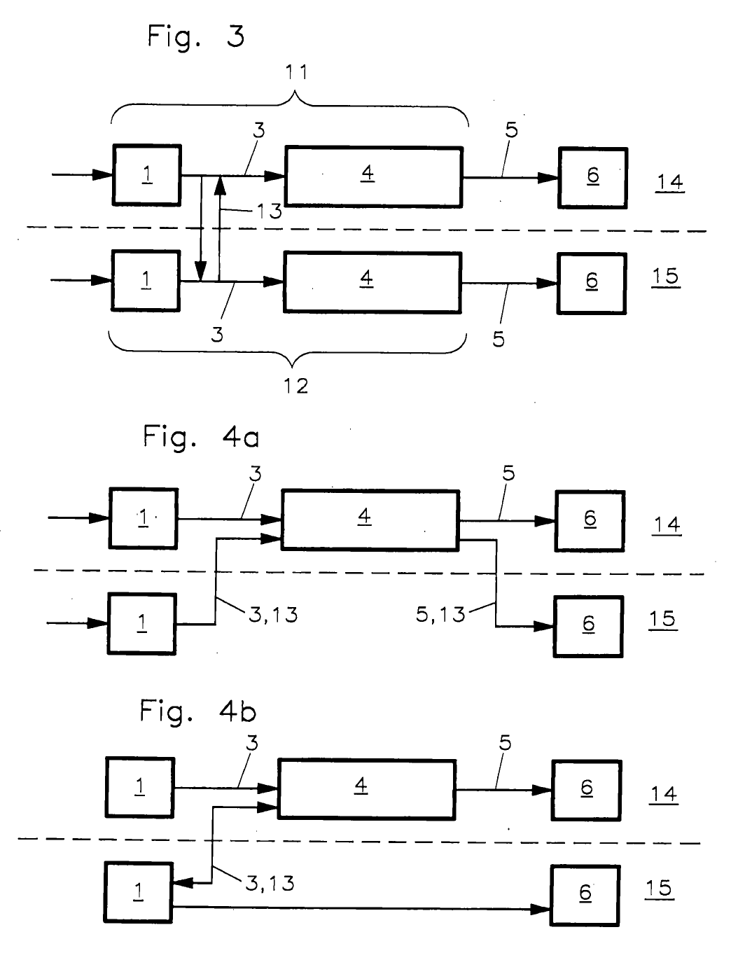

FIG. 3 discloses a first embodiment for binocular usage. In this figure, in addition to the first system 11 in or on the first eye 14, there is also a second OTS having second system 12 for controlling the accommodation demand detection in or on the second eye 15, comprising at least one sensor 1 for detecting a signal sequence from the body itself. Signals are thus detected in both eyes. Preferably, the artificial accommodation system or the OTS and, more preferably, the controller for controlling the accommodation demand detection are identical in both eyes. Likewise preferably, the accommodation demand is detected in a redundant manner. For recognizing the signals, unidirectional or bidirectional data exchange 13 takes place between the two systems 11 and 12, preferably of the measurement signals 3 between the sensor 1 and detector 4.

FIG. 4a and b disclose other embodiments for binocular usage. These are distinguished in that the first and second eye each have their own sensor 1 and a separate accommodation system 6 (or OTS), which, however, communicate with a common detector 4. The systems are preferably not redundant. Instead, they divide up the tasks. Preferably, the detector is positioned in one of the two eyes (the first eye 14 in the example) and preferably communicates wirelessly with the sensor and the accommodation system of the other eye (the second eye 15 in the example). While FIG. 4a shows a design in which the common detector 4 directly communicates with the sensor and the accommodation system in the other eye, the design shown in FIG. 4b uses the sensor in the other eye as the single transmitter for data exchange with the central detector. The accommodation system 6 of the second eye 15 thus receives the control signal indirectly via the sensor.

Therefore, binocular usage concepts do not only include concurrent use of two identical controllers for controlling the accommodation demand detection in an ophthalmic technical system which may or may not have the aforementioned data exchange, but also the use of two different systems or one system in particular having different sensors for separate detection of e.g. the lid reflex and eye orientation in different eyes. In this respect, the sensors are preferably used as transmitters and receivers for wireless unidirectional or bidirectional data exchange.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE NUMERALS

1 Sensor

2 Signal sequence

3 Measurement signal

4 Detector

5 Control signal

6 Accommodation system

7 Segmentation

8 Signal detection

9 Trigger

10 Control action

11 First system

12 Second system

13 Data exchange

14 First eye

15 Second eye

1. A control system for controlling an accommodation demand detection in an ophthalmic technical system, the control system comprising:
a) at least one sensor configured to detect a signal sequence from a body or an environmental signal sequence; and to convert the detected signal sequence into measurement signals, and
b) at least one detector configured to convert the measurement signals into a control signal that influences the accommodation demand detection,
wherein the at least one detector comprises a signal processor configured to compares the measurement signals with a reference signal bandwidth and to generates the control signal when the measurement signals are covered by a predetermined reference signal bandwidth.

2. The control system according to claim 1, wherein the signal sequence from the body includes a description of unconscious saccade and/or blinking movements and/or of conscious eye movements, and
wherein the control signal is a trigger signal for the accommodation demand detection.

3. The control system according to claim 1, wherein the signal sequence from the body includes a description of everyday behavior patterns, or the environmental signal sequence includes a description of environmental influences, and
wherein the control signal is an actuation signal for a predetermined focal length range or a predetermined frequency in the accommodation demand detection in the ophthalmic technical system.

4. The control system according to claim 1, wherein the at least one sensors is an acceleration sensor, a magnetic field sensor, and/or a gyroscope.

5. The control system according to claim 4, wherein the measurement signals are acceleration signals or orientation signals and the signal processor detects an acceleration change above an acceleration threshold.

6. The control system according to claim 5, wherein the control signal comprises a trigger signal or a trigger signal sequence for the accommodation demand detection or for adjusting a fixed focal length in the ophthalmic technical system.

7. The control system according to claim 6, wherein the trigger signal sequence comprises at least three trigger signals having an increasing interval there between.

8. The control system according to claim 1, wherein at least one of the sensors is a detector for detecting ambient light and/or blinking.

9. The control system according to claim 1, wherein the at least one sensor includes, two sensors configured to communicate with one another by data exchange.

10. A contact lens comprising:
an ophthalmic technical system; and
a control system for controlling an accommodation demand detection in the ophthalmic technical system, the control system comprising:
a) at least one sensor configured to detect a signal sequence from a body or an environmental signal sequence and to convert the detected signal sequence into measurement signals, and
b) at least one detector configured to convert the measurement signals into a control signal that influences the accommodation demand detection,
wherein the at least one detector comprises a signal processor configured to compare the measurement signals with a reference signal bandwidth and to generate the control signal when the measurement signals are covered by a predetermined reference signal bandwidth.
